(19) United States
(12) Patent Application Publication
(10) Pub. No.: US 2025/0213456 A1
(43) Pub. Date: Jul. 3, 2025

(54) ORAL CARE COMPOSITION COMPRISING A PEROXIDE AND AN ETHYLENE OXIDE/PROPYLENE OXIDE BLOCK COPOLYMER

(71) Applicant: BASF SE, Ludwigshafen am Rhein (DE)

(72) Inventors: Changwen Dennis ZHANG, Tarrytown, NY (US); Joel BASILAN, Tarrytown, NY (US)

(73) Assignee: BASF SE, Ludwigshafen am Rhein (DE)

(21) Appl. No.: 18/875,297

(22) PCT Filed: Jul. 6, 2023
(86) PCT No.: PCT/US2023/026993

§ 371 Date: Dec. 16, 2024

Related U.S. Application Data
(60) Provisional application No. 63/388,052, filed on Jul. 11, 2022.

(51) Int. Cl. A61K 8/90; A61K 8/22; A61K 8/34; A61Q 11/00

(52) U.S. Cl. CPC A61K 8/90; A61K 8/22; A61Q 11/00; A61K 8/345; A61K 2800/28; A61K 2800/54; A61K 2800/596

(57) ABSTRACT

Described herein is an oral care composition. The oral care composition includes an ethylene oxide/propylene oxide block copolymer of the formula HO(C₂H₄O)ₐ(C₃H₆O)_{b}(C₂H₄O)ₐH, wherein per mole of such copolymer there are a total of about 25 to about 50 moles of ethylene oxide units and about 5 to about 11 moles of propylene oxide units, wherein a is from 50 to 400, b is from 50 to 400, and wherein the copolymer has a molecular weigh of about 1250 to about 15000. The oral care composition also includes a peroxide and an emulsifier. The oral care composition includes the ethylene oxide/propylene oxide block copolymer in an amount of at least about 20 wt % based on a total weight of the composition.

### CROSS REFERENCE TO RELATED APPLICATION(S)

The present application claims priority to U.S. Provisional Patent Application No. 63/388,052, filed on Jul. 11, 2022, the entire contents of which are incorporated herein in its entirety.

### FIELD OF THE INVENTION

The present invention relates to an oral care composition.

### BACKGROUND OF THE INVENTION

Peroxide is commonly present in compositions for tooth whitening treatment in both professional and at home applications. However, it is difficult to formulate with high levels of peroxide, as peroxide can be unstable. For example, the oxidative nature of peroxide causes many challenges for oral care compositions such as toothpaste, gel and mouthwash. That is, when peroxide breaks down, it releases oxygen, water and radicals. The excess gas can cause swelling and bursting of product packaging and the radicals can cause the entire composition, including the actives and flavors, to break down to a composition with a waterlike viscosity and decreased efficacy.

To address this, one strategy to stabilize the peroxide is to reduce the amount of water and use solid forms of peroxide. However, there are several limitations associated with low water/anhydrous compositions. For example, conventional thickeners such as xanthan gum require water hydration to reach full gelling capability. Another limitation is that the solid content of the low water/anhydrous composition is high. Increasing the solid peroxide content is very challenging because a higher amount of solid peroxide means more solids, higher viscosity and less flowability.

Thus, there is a need to improve the oral care composition including a high level of peroxide.

### SUMMARY OF THE INVENTION

It has been found that using an ethylene oxide/propylene oxide block copolymer, and an emulsifier in combination with a peroxide enables the use of a liquid peroxide and removes the restriction of anhydrous formulation.

In an embodiment, an oral care composition is provided. The oral care composition includes:
an ethylene oxide/propylene oxide block copolymer of the formula:

HO(C₂H₄O)ₐ(C₃H₆O)_{b}(C₂H₄O)ₐH
wherein per mole of such copolymer there are a total of about 25 to about 50 moles of ethylene oxide units and about 5 to about 11 moles of propylene oxide units, wherein a is from 50 to 400, b is about 50 to 400, and the copolymer has a molecular weight of from about 1250 to 15000 kg/mol; a peroxide, and an emulsifier, wherein the ethylene oxide/propylene oxide block copolymer is in an amount of at least 20 wt % based on a total weight of the composition.

In another embodiment, a method of whitening includes applying to a surface of a mammalian tooth an oral care composition.

### DETAILED DESCRIPTION OF THE INVENTION

Oral care compositions can include fluoride, peroxide, abrasives, flavors and other ingredients to provide benefits like reducing plaque and tartar, preventing cavities, preventing and reversing gingivitis, building protection against sensitivity, freshening bad breath, and whitening teeth. Some consumers are particularly interested in a product that contains a relatively high amount of peroxide, for instance greater than 1%, in order to enhance removal of intrinsic and extrinsic stains.

However, oral care compositions, especially those containing a peroxide source, can be unstable and difficult to formulate. For example, peroxides readily decompose, such as hydrogen peroxide which decomposes to form water and oxygen. When peroxide decomposes it can be less efficacious, produce excess gas, which can cause swelling and bursting of primary packaging, and can impact the structure stability and efficacy of the other components in the oral care composition, including flavors. To address this, many oral care compositions including a solid form of peroxide are anhydrous or have a low water content. However, such formulations have limitations at high concentration of peroxide due to the high solid content.

Embodiments of the present invention advantageously provide an oral care composition including an aqueous peroxide for easier application of the oral care composition, and to save cost during manufacturing. Embodiments further advantageously utilize a component in the oral care composition that can help stabilize peroxide and maintain the original viscosity. In an embodiment, the oral care composition may include an ethylene oxide/propylene oxide block copolymer, a surfactant and a peroxide. The viscosity of the composition may be about 1,000 cP to about 1,000,000 cP.

In one embodiment, the oral care composition may include an ethylene oxide/propylene oxide block copolymer of the formula:

HO(C₂H₄O)ₐ(C₃H₆O)_{b}(C₂H₄O)ₐH
wherein per mole of such copolymer there are a total of about 25 to about 50 moles of ethylene oxide units and about 5 to about 11 moles of propylene oxide units, and said copolymer has a molecular weight of from about 1250 to 15000 kg/mol, an emulsifier, and a peroxide. In the formula of the ethylene oxide/propylene oxide block copolymer, a and b may be from 50 to 400.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The articles “a”, “an”, and “the” are used herein to refer to one or to more than one (i.e., to at least one) of the grammatical object of the article. By way of an example, “a filter” means one filter or more than one filter.

Unless explicitly stated otherwise, all percentages stated in connection with the compositions herein described refer to a weight percent, respectively based on the mixture or composition in question.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The composition can contain, consist of, or consist essentially of, the essential elements and limitations of the invention described herein, as well as any additional or optional ingredients, components, or limitations described herein or otherwise useful in oral care compositions.

As used herein, “oral care composition” is meant as a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, mouth rinse, mousse, foam, mouth spray, lozenge, chewable tablet, chewing gum, tooth whitening strips, whitening gels, toothpaste, floss and floss coatings, breath freshening dissolvable strips, denture care, or adhesive product. The oral care composition may also be incorporated into strips or films for direct application or attachment to oral surfaces.

As used herein, the term “dentifrice” includes tooth or subgingival paste, gel or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

As used herein, the term “dispenser” refers to any pump, tube or container suitable for dispensing compositions such as dentifrices.

As used herein, the term “teeth” refers to natural teeth, artificial teeth or dental prosthesis.

In an embodiment of the present disclosure, an oral care composition is provided. The oral care composition may include an ethylene oxide/propylene oxide block copolymer. The oral care composition may include an ethylene oxide/propylene oxide block copolymer of the formula:

HO(C₂H₄O)ₐ(C₃H₆O)_{b}(C₂H₄O)ₐH
wherein per mole of such copolymer there are a total of about 25 to about 50 moles of ethylene oxide units and about 5 to about 11 moles of propylene oxide units, and said copolymer has a molecular weight of from about 1250 to 15000 kg/mol, an emulsifier, and a peroxide. In some embodiments, the copolymer may have a molecular weight of from about 1500 to about 14500 kg/mol, about 1750 to about 14000 kg/mol, about 2000 to about 13500 kg/mol, about 2250 to about 13000 kg/mol, about 2500 to about 12500 kg/mol, about 2750 to about 12000 kg/mol, about 3000 kg/mol to about 11500 kg/mol, about 3500 to about 11000 kg/mol, about 4000 kg/mol to about 10500 kg/mol, about 4500 to about 10000 kg/mol, about 5000 to about 9500 kg/mol, about 5500 to about 9000 kg/mol, about 6000 to about 8500 kg/mol, or about 7500 to about 8000 kg/mol, or any range or sub value herein. The ethylene oxide/propylene oxide block copolymer may be included in the oral care composition in an amount of at least about 20 wt % based on a total weight of the composition. It has been found that when the amount of ethylene oxide/propylene oxide block copolymer is less than about 20 wt %, the oral care composition does not form a stable structure.

In some embodiments, the emulsifier may include an anionic emulsifier, a non-ionic emulsifier or a combination thereof. The emulsifier may be stearyl alcohol, cetearyl alcohol, cetyl alcohol, stearyl and sodium lauryl sulfate, sodium lauryl sulfate, sodium lauryl glucose carboxylate, lauryl glucoside, or a combination thereof.

It was found that including an emulsifier in the oral care composition balanced the viscosity and texture of the composition.

In an embodiment of the present disclosure, the peroxide may include, but is not limited to, hydrogen peroxide, urea peroxide, calcium peroxide, sodium peroxide, zinc peroxide or combinations thereof. In one embodiment of the oral care composition, the peroxide may include hydrogen peroxide. In some embodiments, the amount of peroxide in the oral care composition may be about 0.01 wt %, about 0.5 w %, at least about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, or within any range defined therebetween (e.g., from about 1.5% to about 7.5%). In another embodiment, the amount of peroxide in the oral composition is about 0.5% to about 10%, about 1% to about 9%, about 2% to about 8%, about 3% to about 7%, or about 4% to about 6%.

In some embodiments, the peroxide may be a liquid formulation. The concentration of peroxide in the liquid formulation may be about 20 wt %, about 25 wt %, about 30 wt %, about 35 wt %, about 40 wt %, about 45 wt %, or about 50 wt %, or within any range defined therebetween (e.g., from about 20 wt % to about 40 wt %).

In some embodiments, the oral care composition may further include water. The oral care composition may have a water concentration of about 5% to about 20%. In some embodiments, the water concentration may be about 5%, about 7%, about 10%, about 12%, about 15%, about 17% or about 20%. In other embodiments, the water concentration may be from about 1% to about 25%, from about 5% to about 20%, from about 7% to about 18%, or from about 10% to about 15%, or any value or subrange herein.

In some embodiments, the oral care composition may include at least one excipient. Excipients suitable for use include any compound that is conventionally used in oral care compositions. Suitable excipients for an oral composition may be selected from preservatives, abrasives (smoothing agents), antibacterial agents, inflammation-inhibiting agents, irritation-preventing agents, irritation-inhibiting agents, antimicrobial agents, antioxidants, binders, (mineral) fillers, buffers, carrier materials, chelating agents, cleaning agents, care agents, surface-active substances, enzymes, foam-forming agents, foam stabilizers, foam boosters, gelling agents, gel-forming agents, bleaching agents, smell- and/or taste-modulating agents, smell- and/or taste reducing agents, smell and/or taste enhancing agents, plasticizers, skin cooling agents, skin soothing agents, skin cleansing agents, skin care agents, skin healing agents, mucous membrane-protecting agents, stabilizers, suspending agents, vitamins, colorants, color-protecting agents, pigments, surfactants, electrolytes, silicone derivatives, polyols, calcium carbonate, calcium hydrogen phosphate, aluminum oxide, fluorides, zinc, tin, potassium, sodium and strontium salts, pyrophosphates, or hydroxyapatites.

In some embodiments, an oral care composition in accordance with the present invention includes at least one excipient, wherein the at least one excipient is chosen from: surfactants, desensitizing agents, whitening agents, tartar control agents, antibacterial agents, abrasives, chelants including silica, binders and thickening agents, detergents, adhesion agents, foam modulators, pH modifying agents, mouth-feel agents, sweeteners, flavoring agents, colorants, preservatives, combinations thereof, and the like.

In some embodiments, the oral care composition may further include a surfactant. In some embodiments of the present disclosure, the surfactant may be an anionic surfactant, a non-ionic surfactant or a combination thereof. In some embodiments, the surfactant may also include zwitterionic, amphoteric, or cationic surfactants. In some embodiments, the anionic surfactant may be sodium lauryl sulfate, sodium lauryl glucose carboxylate, lauryl glucoside, or a combination thereof. In some embodiments, the non-ionic surfactant may include an alkyl polyglucoside, or lauryl glucoside. In some embodiments, the surfactant may be sodium lauryl glucose carboxylate and lauryl glucoside.

The surfactant may be prepared in a solvent. The solvent may include water, edible polyhydric alcohol such as glycerin, diglycerin, triglycerin, sorbitol, xylitol, butylene glycol, erythritol, polyethylene glycol, propylene glycol, or combinations thereof.

In some embodiments, the surfactant may include anionic surfactants such as organophosphate, which include alkyl phosphates, alkyl or alkenyl phosphate esters. The organophospate surfactant has a strong affinity for enamel surface and sufficient surface binding propensity to desorb pellicle proteins and remain affixed to enamel surfaces. Examples of suitable surfactants include alkyl and alkyl (poly)alkoxy phosphates such as lauryl phosphate; PPG5 ceteareth-10 phosphate; Laureth-1 phosphate; Laureth-3 phosphate; Laureth-9 phosphate; Trilaureth-4 phosphate; C₁₂-C₁₈ PEG 9 phosphate; Sodium dilaureth-10 phosphate. In one example, the alkyl phosphate is polymeric. Examples of polymeric alkyl phosphates include those containing repeating alkoxy groups as the polymeric portion, in particular 3 or more ethoxy, propoxy, isopropoxy or butoxy groups.

Zwitterionic or amphoteric surfactants may include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, such as carboxy, sulfonate, sulfate, phosphate or phosphonate. Amphoteric surfactants may include amine oxide surfactants.

Additional suitable polymeric organophosphates may include dextran phosphate, polyglucoside phosphate, alkyl polyglucoside phosphate, polyglyceryl phosphate, alkyl polyglyceryl phosphate, polyether phosphates and alkoxylated polyol phosphates. Some examples include polyethylene glycol (PEG) PEG phosphate, polypropylene glycol (PPG) phosphate, alkyl PPG phosphate, PEG/PPG phosphate, alkyl PEG/PPG phosphate, PEG/PPG/PEG phosphate, dipropylene glycol phosphate, PEG glyceryl phosphate, polybutylene glycol (PBG) phosphate, PEG cyclodextrin phosphate, PEG sorbitan phosphate, PEG alkyl sorbitan phosphate, and PEG methyl glucoside phosphate. Suitable non-polymeric phosphates include alkyl mono glyceride phosphate, alkyl sorbitan phosphate, alkyl methyl glucoside phosphate, and alkyl sucrose phosphates.

In some embodiments, the oral care composition may further include an abrasive. The abrasive may include silica, such as a gel or precipitate, alumina, phosphates including orthophosphates, polymetaphosphates, pyrophosphates, or combinations thereof. Specific example may include silicone microspheres such as polyorganosilsequioxane particles, dicalcium orthophosphate dihydrate, calcium pyrophosphate, tricalcium phosphate, calcium polymetaphosphate, insoluble sodium polymetaphosphate, rice hull silica, hydrated alumina, beta calcium pyrophosphate, calcium carbonate, and resinous abrasive materials such as particulate condensation products of urea and formaldehye. In some embodiments, the abrasive may be silica, polyorganoosilsquioxane, calcium pyrophosphate, poly (methyl methacrylate), calcium carbonate, dicalcium phosphate, barium sulfate or combinations thereof.

In some embodiments, the amount of abrasive included in the oral care composition may be about 20 wt % based on the total weigh of the oral care composition. In some embodiments, the amount of abrasive included may be about 10 wt % to about 30 wt %, about 12 wt % to about 28 wt %, about 15 wt % to about 25 wt %, or about 17 wt % to about 22 wt %

The oral care composition may have a viscosity of about 1,000 to about 1,000,000 cP, measured with a Brookfield® Viscometer HA, spindle V75, measuring speed 5 rpm. In some embodiments, the viscosity may be about 1,000 cP, about 10,000 cP, about 25,000 cP, about 50,000 cP, about 75,000 cP, about 100,000 cP, about 125,000 cP, about 150,000 cP, about 175,000 cP, about 200,000 cP, about 250,000 cP, about 275,000 cP, about 300,000 cP, about 325,000 cP, about 350,000 cP, about 375,000 cP, about 400,000 cP, about 425,000 cP, about 450,000 cP, about 475,000 cP, about 500,000 cP, about 525,000 cP, about 550,000 cP, about 575,000 cP, about 600,000 cP, about 625,000 cP, about 650,000 cP, about 675,000 cP, about 700,000 cP, about 725,000 cP, about 750,000 cP, about 775,000 cP, about 800,000 cP, about 825,000 cP, about 850,000 cP, about 875,000 cP, about 900,000 cP, about 925,000 cP, about 950,000 cP, about 975,000 cP, or about 1,000,000 cP.

In some embodiments of the oral care composition, after 4 weeks at 40° C. at least about 80% of the peroxide remains, at least about 85% of the peroxide remains, at least about 90% of the peroxide remains, at least about 91% of the peroxide remains, at least about 92% of the peroxide remains, at least about 93% of the peroxide remains, at least about 94% of the peroxide remains, at least about 95% of the peroxide remains, at least about 96% of the peroxide remains, at least about 97% of the peroxide remains, at least about 98% of the peroxide remains, or at least about 99% of the peroxide remains according to the Peroxide stability test.

The Peroxide stability test is performed using the following method to determine the stability of the peroxide in an oral care composition. First, 0.4000 g (+/−0.02 g) of the oral care sample is added to a 250 ml plastic beaker. This procedure is performed in duplicate. Then, 100 mL of 0.04N H₂SO₄ (sulfuric acid) is added to the beaker. A stir bar is added, the beaker is covered with a plastic paraffin film such as Parafilm M® (available from Bemis® Flexible Packaging, Oshkosh, Wisconsin), and the solution is stirred for at least ten minutes (or much longer) until the sample looks homogeneous. After stirring, 25 mL of 10% KI (potassium iodide) solution and 3 drops of NH4-Molybdate are added to the beaker, the beaker is covered with the plastic paraffin film, and the solution is stirred for another 3-20 minutes. The resulting solution is analyzed via autotitration with 0.1N sodium-thiosulfate solution. The % hydrogen peroxide is calculated using the following formula:

$$\% Hydrogen Peroxide = \frac{Thiosulfate used ( mL ) * Thiosulfate Normality ( \frac{m e q}{m L} ) * 1.701 g / meq}{Sample Weight ( g )}$$

Measurements were taken after the initial making of the formulation and then again after the formulation was stored in a non-reactive vessel for a given time period at 40° C. Peroxide stability was calculated as the peroxide percent measured after a given time period at 40° C. divided by the initial peroxide percent measured, then multiplied by 100. Product placed at 40° C. represents accelerated stability and can be used to determine how the oral care compositions may react throughout their shelf life. The given time can be any suitable amount of time. For example, four weeks, eight weeks, 13 weeks or 26 weeks.

In other embodiments, the oral care composition may also include at least one ingredient from the group consisting of desensitizing agents, chelating agents, whitening agents, tartar control agents, detergents, adhesion agents, foam modulators, pH modifying agents, mouth-feel agents, sweeteners, flavoring agents, colorants, preservatives, humectant and combinations thereof.

In some embodiments, the oral care composition may include a flavoring agent. The flavoring agent is a member chosen from: mucous membrane cooling agents, mucous membrane warming agents, sharp-tasting substances, sweeteners, sugar substitutes, organic or inorganic acidifiers (e.g., malic acid, acetic acid, citric acid, tartaric acid, phosphoric acid), bitter principles (e.g., quinine, caffeine, limonine, amarogentine, humolones, lupolones, catechols, tannins), edible mineral salts (e.g., sodium chloride, potassium chloride, magnesium chloride and sodium phosphates), essential oils (e.g., oils of spearmint, peppermint, wintergreen, sassafras, clove, sage, eucalyptus, marjoram, cinnamon, lemon, lime, grapefruit, and orange), menthol, carvone, anethole, and combinations thereof.

Anti-microbial agents may include nonionic antibacterial agents, including halogenated diphenyl ether compounds such as 2,4,4′-trichloro-2′-hydroxydiphenyl ether (Triclosan) and 2,2′-dihydroxy-5,5′-dibromodiphenyl ether. Other useful nonionic antibacterial agents may include phenolic compounds including phenol and its homologs, mono and polyalkyl and aromatic halophenols, resorcinol and its derivatives and bisphenolic compounds, such phenolic compounds being more fully disclosed in U.S. Pat. No. 5,368,844, the disclosure of which is incorporated herein by reference.

In some embodiments, an oral care composition in accordance with the present disclosure includes an anti-microbial agent. In some embodiments, the anti-microbial agent is a member chosen from: triclosan, chlorhexidine and its salts (e.g., its acetate, gluconate or hydrochloride), peroxides, phenols and their salts, domiphen bromide (phenododecinium bromide), bromochlorophene, Zn salts, stannous fluoride, chlorophylls, Cu salts, Cu gluconate, Cu chlorophyll, sodium lauryl sulfate, polylysine, quarternary monoammonium salts such as cocoaliphaticbenzyldimethylammonium chloride, homopolymers of arginine, arginine salts or complexes, or also pyridinium salts such as cetyl pyridinium chloride, and combinations thereof.

Thymol, menthol, methyl salicylate and eucalyptol and mixtures thereof are well known as antimicrobials and active against plaque.

In some embodiments, the anti-microbial agent is a nonionic antibacterial agent. In some embodiments, the nonionic antibacterial agent is included in a oral care composition at a concentration of about 0.001 wt. % to about 5 wt. %. In some embodiments, the nonionic antibacterial agent is present in an amount of about 0.01 wt. % to about 1.5 wt. %.

Anti-microbial agents of particular interest are quarternary monoammonium salts such as cocoaliphaticbenzyldimethylammonium chloride or also pyridinium salts such as cetyl pyridinium chloride, polylysine, thymol, menthol, methyl salicylate and eucalyptol triclosan, chlorhexidine and its salts (e.g., its acetate, gluconate or hydrochloride) and combinations thereof.

In some embodiments, an oral care composition may include an anti-caries agent. In some embodiments, the anti-caries agent is a fluoride ion source chosen from: inorganic fluoride salts, such as soluble alkali metal, alkaline earth metal salts (e.g., sodium fluoride, stannous fluoride, potassium fluoride, ammonium fluoride, calcium fluoride), a copper fluoride such as cuprous fluoride, zinc fluoride, barium fluoride, sodium fluorosilicate, ammonium fluorosilicate, sodium fluorozirconate, ammonium fluorozirconate, sodium monofluorphosphate, aluminum mono- and di-fluorophosphate, fluorinated sodium calcium pyrophosphate, and combinations thereof.

In some embodiments, an oral care composition may include an anti-tartar agent. In some embodiments, the anti-tartar agent is chosen from: pyrophosphate salts including dialkali or tetraalkali metal pyrophosphate salts such as Na₄P₂O₇, K₄P₂O₇, Na₂K₂P₂O₇, Na₂H₂P₂O₇ and K2H2P2O7, sodium tripolyphosphate; long chain polyphosphates such as sodium hexametaphosphate; and cyclic phosphates such as sodium trimetaphosphate. In some embodiments, an anti-tartar agent is present in an oral care composition at a concentration of about 1 wt. % to about 5 wt. %.

In some embodiments, the oral care composition may further contain desensitizing agents. For example nitrate salts, a bicarbonate salts, potassium nitrate, arg/n/ne-bicarbonate-phytate complex, potassium citrate or oxalate and arginine.

In some embodiments, an oral care composition may include a thickening agent. In some embodiments, the thickener is selected from the group consisting of, but not limited to: calcium carbonate, titanium dioxide, silicon dioxide, talcum, aluminium oxide, dicalcium phosphate, tricalcium phosphate, magnesium hydroxide, cellulose thickeners such as carboxymethyl cellulose, hyroxyalkyl celluloses such as hydroxypropyl cellulose hydroxyethyl cellulose, gums such as xanthan gum, polyglycols and polyethylene glycol, inorganic thickeners (e.g., amorphous silica compounds, natural and synthetic clays, lithium magnesium silicate and magnesium aluminum silicate), and combinations thereof.

In some embodiments, the thickening agent is an organic thickener chosen from: natural and synthetic gums and colloids including cellulose thickeners such as carboxymethyl cellulose; hyroxyalkyl celluloses such as hydroxypropyl cellulose hydroxyethyl cellulose; gums such as xanthan gum; polyglycols of varying molecular weights sold under the tradename Polyox™; and polyethylene glycol. In some embodiments, the thickening agent is an inorganic thickener chosen from: amorphous silica compounds such as colloidal silicas compounds available under the trade designation Cab-o-Sil® (manufactured by Cabot Corporation and distributed by Lenape Chemical, Bound Brook, N.J.); Zeodent® 165 (J.M. Huber Chemicals Division, Havre de Grace, Md. 21078); Sylodent® 15 (Davison Chemical Division of W.R. Grace Corporation, Baltimore, Md. 21203); natural and synthetic clays; lithium magnesium silicate (Laponite); and magnesium aluminum silicate (Veegum). In some embodiments, the thickening agent is present in an oral care composition in amounts of about 0.1 wt. % to about 10 wt. %. In some embodiments, the thickening agent is present in an amount of about 0.5 wt. % to about 4.0 wt. %.

In some embodiments, an oral care composition may include an anti-oxidant. In some embodiments, the anti-oxidant is chosen from: naturally occurring tocopherols and their derivatives (e.g., Vitamin E acetate), Vitamin C and its salts and derivatives (e.g., ascorbyl palmitate, Mg ascorbyl phosphate, ascorbyl acetate), Vitamin A and derivatives (Vitamin A palmitate), tocotrienols, flavonoids, alpha-hydroxy acids (e.g., citric acid, lactic acid, malic acid, tartaric acid) and their Na, Ka and Ca salts, flavonoids, quercetin, phenolic benzylamines, propyl gallate, octyl gallate, dodecyl gallate, butylhydroxyanisole (BHA, E320), butylhydroxytoluene (BHT, 2,6-di-tert.-butyl-4-methylphenol, E321), lecithins, mono- and diglycerides of edible fatty acids esterified with citric acid, carotenoids, carotenes (e.g., o carotene, β-carotene, lycopene) and their derivatives, phytic acid, lactoferrin, EDTA, EGTA), folic acid and its derivatives, ubiquinone and ubiquinol and their derivatives, ferulic acid and its derivatives, zinc and its derivatives (e.g., ZnO, ZnSO₄), selenium and its derivatives (e.g., selenium methionine), orthophosphates and Na, K and Ca salts of mono-phosphoric acids, and constituents, extracts and fractions thereof isolated from plants, (e.g., tea, green tea, algae, grapeseeds, wheat germ, chamomile, rosemary, oregano), and combinations thereof.

Antioxidants of particular interest are octadecyl 3-(2,5-di-tert-butyl-4-hydroxyl phenyl propionate, tetrabutyl ethylidinebisphenol and tetrakis (3-(3,5-di-tert-butyl-4-hydroxyphenyl)propionate.

Light stabilizers may also be included in the oral care compositions. A good example of a Benzotriazole light stabilizers is 2-(5-chloro-2H-benzotriazol-2-yl)-6-(1, 1 dimethylethyl)-4-methyl phenol.

The oral care composition of the present disclosure may be a product selected from the group consisting of orally dissolvable films, whitening strips, whitening gels, mouthwashes, tooth pastes or dentifrices.

In an embodiment of the present disclosure, a method for whitening is also provided. The method includes applying an oral care composition as described herein to a surface of a mammalian tooth.

### EXAMPLES

The present invention will now be more fully described with reference to the accompanying examples. It should be understood, however, that the following description is illustrative only and should not be taken in any way as a restriction of the invention.

### Example 1

An oral care composition in accordance with the present disclosure was prepared. An emulsifier, Lanette W®, was melted and then added to water at 65° C. The mixture is mixed until it became a viscous white paste. Tetrasodium pyrophosphate (TSPP), sodium saccharin and sodium monofluorophosphate was added to 142 g of hydrogen peroxide and mixed until dissolved to form a peroxide solution. A ethylene oxide/polypropylene oxide block copolymer (L1220®) and the surfactant paste was added to a Ross Mixer and mixed. Then, the peroxide solution was added to the Ross Mixer and mixed. Next, calcium pyrophosphate was added to the mixture, which was then mixed. Additional excipients were then added and mixed to form the final formulation, shown in Table 1 below.

TABLE 1
Oral Care Composition Formulation

| | | 1 kg batch |
|---|---|---|
| Ingredients | Weight % | (g) |
| Water | 12.57 | 125.74 |
| Calcium Pyrophoshpate | 20.00 | 200.00 |
| Pluracare L1220 | 20.00 | 200.00 |
| Glycerin | 20.00 | 200.00 |
| Hydrogen Peroxide (35% solution) | 14.29 | 142.86 |
| Plantaren 1200 N UP | 3.00 | 30.00 |
| Plantaren2000 N UP | 1.00 | 10.00 |
| Flavor | 1.50 | 15.00 |
| TSPP | 1.00 | 10.00 |
| Sodium Saccharin | 0.50 | 5.00 |
| Sodium Monofluorophosphate | 1.14 | 11.40 |
| Lanette W | 5.00 | 50.00 |

The initial viscosity of the oral care composition was measured to be 521,400 cP and had an initial pH of 7.75. A visual inspection was conducted and the paste was smooth.

### Example 2

A peroxide stability test was also conducted. To determine the peroxide stability, a sample was added and weighed (to the nearest 0.1 mg) in a 150 ml beaker for (w/w) analysis or the volume was measured using volumetric pipette for (w/v) analysis according to the equation below targeting approximately 8 mL of titrant. The sample weight should be no less than approximately 0.1 g and no more than approximately 12 g sample.

$$Sample weight ( g ) = ( 8 mL sodium thiosulfate ) * ( 0.1 N ) * ( meq wt of oxidizing agent ) * \frac{100}{\% oxidizing agent expeted}$$

For rinses, add approximately 50 mL of water. For gels, dentifrices, tooth whiteners and other samples containing high levels of surfactants or high organic content, add 50 mL of ethanol/water (1:1 v/v) solution. Mix until the sample dispersed. For samples that do not disperse in water, i.e. polymer based formulas, add approximately 30 mL of chloroform in order to disperse the polymer matrix. Then proceed with the addition of approximately 50 mL of ethanol/water (1:1 v/v) solution.

25 mL of glacial acetic add was slowly added to the mixture followed immediately by 5 mL of 20% potassium iodide solution and 4 drops of ammonium molybdate solution. The solution was mixed for 5 minutes. 2 mL of starch indicator solution was added and a dark blue-black color appeared, then the solution was titrated with sodium thiosulfate 0.1N solution while mixing the sample until the dark blue-black color disappeared. Some residual color may still remain. If so, the titration should slowly continue until the end point has been reached and no color is present. The percent of oxidizing agent is then calculated according to the following formula.

$$\% Oxidizing Agent = \frac{( mL sodium thiosulfate ) * N * ( meq wt of oxidizing agent ) * 100}{Sample Weight ( gms ) or Sample Volume ( mL )}$$

Where: N=Normality of Sodium Thiosulfate solution
   meq=0.00800 for active oxygen
   meq=0.04704 for urea peroxide
   meq=0.01701 for hydrogen peroxide
   meq=0.03604 for calcium peroxide

A 5% peroxide toothpaste formulation including a high amount of surfactant was prepared in accordance with the oral care composition of the present disclosure. A peroxide stability test was then conducted at the following conditions in Table 2. The results of which are also presented in Table 2.

TABLE 2
Peroxide Stability Test

| Sample | Time | Temperature | % Peroxide |
|---|---|---|---|
| 5% peroxide | Initial | RT | 5.071 |
| toothpaste of | | | |
| present disclosure | | | |
| 5% peroxide | 4 weeks | RT | 4.479 |
| toothpaste of | | | |
| present disclosure | | | |
| 5% peroxide | 4 weeks | 40° C. | 4.293 |
| toothpaste of | | | |
| present disclosure | | | |
| Aqueous | Initial | RT | 3.628 |
| Comparative | | | |
| Example | | | |
| (4% peroxide) | | | |
| Aqueous | 2 weeks | 40° C. | 3.620 |
| Comparative | | | |
| Example | | | |
| (4% peroxide) | | | |
| Aqueous | 4 weeks | 40° C. | 3.615 |
| Comparative | | | |
| Example | | | |
| (4% peroxide) | | | |
| Anhydrous | Initial | RT | 4.508 |
| Comparative | | | |
| Example | | | |
| (5% peroxide) | | | |
| Anhydrous | 2 weeks | 40° C. | 4.557 |
| Comparative | | | |
| Example | | | |
| (5% peroxide) | | | |
| Anhydrous | 4 weeks | 40° C. | 4.253 |
| Comparative | | | |
| Example | | | |
| (5% peroxide) | | | |

From this test, it can be seen that the peroxide stability of the toothpaste formulation of the present disclosure had similar stability to the bench mark comparison of the Aqueous Comparative Example 4% and Anhydrous Comparative Example 5% formulation.

In an embodiment, an oral care composition is provided. The oral care composition may include:
an ethylene oxide/propylene oxide block copolymer of the formula:

HO(C₂H₄O)ₐ(C₃H₆O)_{b}(C₂H₄O)ₐH
wherein per mole of such copolymer there are a total of about 25 to about 50 moles of ethylene oxide units and about 5 to about 11 moles of propylene oxide units, wherein a is from 50 to 400, b is about 50 to 400, and the copolymer has a molecular weight of from about 1250 to 15000 kg/mol,
a peroxide, and
an emulsifier;
wherein the ethylene oxide/propylene oxide block copolymer is in an amount of at least 20 wt % based on a total weight of the composition.

In certain embodiments, the emulsifier of the oral care composition may include an anionic emulsifier or non-ionic emulsifier.

In certain embodiments, the emulsifier may include stearyl alcohol, cetearyl alcohol, cetyl alcohol, stearyl alcohol and sodium lauryl sulfate, sodium lauryl glucose carboxylate, lauryl glucoside, or a combination thereof.

In certain embodiments, the peroxide may be included in an amount of about 0.01 wt % to about 10 wt % in the oral care composition.

In certain embodiments, the oral care composition may further include an abrasive. In certain embodiments, the abrasive may include silica, polyorganosilsequioxane, calcium pyrophosphate, poly(methyl methacrylate), calcium carbonate, dicalcium phosphate, barium sulfate, or combinations thereof.

In certain embodiments, the oral care composition may further include a surfactant. In certain embodiments, the surfactant may include an anionic surfactant, a non-ionic surfactant or a combination thereof. In certain embodiments, wherein the non-ionic surfactant includes an alkyl polyglucoside surfactant. In certain embodiments, the anionic surfactant may include sodium lauryl glucose carboxylate and lauryl glucoside.

In certain embodiments, the oral care composition may further include water. In certain embodiments, the composition may have a water concentration of about 5% to about 20%.

In certain embodiments of the oral care composition, wherein after 4 weeks at 40° C. at least about 80% of the peroxide remains, at least about 85% of the peroxide remains, at least about 90% of the peroxide remains, at least about 91% of the peroxide remains, at least about 92% of the peroxide remains, at least about 93% of the peroxide remains, at least about 94% of the peroxide remains, at least about 95% of the peroxide remains, at least about 96% of the peroxide remains, at least about 97% of the peroxide remains, at least about 98% of the peroxide remains, or at least about 99% of the peroxide remains according to the Peroxide stability test.

In certain embodiments, the oral care composition may be a product selected from the group consisting of orally dissolvable films, whitening strips, whitening gels, mouthwashes, tooth pastes, or dentifrices.

In certain embodiments, the composition may have a viscosity of about 1,000 to about 250,000 cP.

In certain embodiment of the oral care composition, the peroxide may include hydrogen peroxide, urea peroxide, calcium peroxide, sodium peroxide, zinc peroxide, or combinations thereof.

In certain embodiments of the oral care composition, the peroxide may be hydrogen peroxide.

In certain embodiments, the oral care composition may include at least one ingredient selected from the group consisting of desensitizing agents, chelating agents, whitening agents, tartar control agents, detergents, adhesion agents, foam modulators, pH modifying agents, mouth-feel agents, sweeteners, flavoring agents, colorants, preservatives, humectant and combinations thereof.

In certain embodiments, the oral care composition may include the humectant. In certain embodiments, the humectant may include glycerin.

In certain embodiments of the oral care composition, the peroxide may be a liquid formulation. In certain embodiments, the liquid formulation may have a concentration of the peroxide of about 20 wt %, about 25 wt %, about 30 wt %, about 35 wt %, about 40 wt %, about 45 wt % or about 50 wt %.

In certain embodiments, a method of whitening is provided. The method of whitening may include applying to a surface of a mammalian tooth the oral care composition according to the present disclosure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a value disclosed as “5 wt %” is intended to mean “about 5 wt %.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oral care composition comprising:
an ethylene oxide/propylene oxide block copolymer of the formula:
HO(C₂H₄O)ₐ(C₃H₆O)_{b}(C₂H₄O)ₐH
wherein per mole of such copolymer there are a total of about 25 to about 50 moles of ethylene oxide units and about 5 to about 11 moles of propylene oxide units, wherein a is from 50 to 400, b is about 50 to 400, and the copolymer has a molecular weight of from about 1250 to 15000 kg/mol,
a peroxide, and
an emulsifier;
wherein the ethylene oxide/propylene oxide block copolymer is in an amount of at least 20 wt % based on a total weight of the composition.

2. The oral care composition of claim 1, wherein the emulsifier comprises an anionic emulsifier or non-ionic emulsifier.

3. The oral care composition of claim 1, wherein the emulsifier comprises stearyl alcohol, cetearyl alcohol, cetyl alcohol, stearyl alcohol and sodium lauryl sulfate, sodium lauryl glucose carboxylate, lauryl glucoside, or a combination thereof.

4. The oral care composition of claim 1, wherein the peroxide is included in an amount of about 0.01 wt % to about 10 wt %.

5. The oral care composition of claim 1, further comprising an abrasive.

6. The oral care composition of claim 5, wherein the abrasive comprises silica, polyorganosilsequioxane, calcium pyrophosphate, poly(methyl methacrylate), calcium carbonate, dicalcium phosphate, barium sulfate, or combinations thereof.

7. The oral care composition of claim 1, further comprising a surfactant.

8. The oral care composition of claim 7, wherein the surfactant comprises an anionic surfactant, a non-ionic surfactant or a combination thereof.

9. The oral care composition of claim 8, wherein the non-ionic surfactant includes an alkyl polyglucoside surfactant.

10. The oral care composition of claim 9, wherein the anionic surfactant comprises sodium lauryl glucose carboxylate and lauryl glucoside.

11. The oral care composition of claim 1, further comprising water.

12. The oral care composition of claim 11, wherein the composition has a water concentration of about 5% to about 20%.

13. The oral care composition of claim 1, wherein after 4 weeks at 40° C. at least about 80% of the peroxide remains, at least about 85% of the peroxide remains, at least about 90% of the peroxide remains, at least about 91% of the peroxide remains, at least about 92% of the peroxide remains, at least about 93% of the peroxide remains, at least about 94% of the peroxide remains, at least about 95% of the peroxide remains, at least about 96% of the peroxide remains, at least about 97% of the peroxide remains, at least about 98% of the peroxide remains, or at least about 99% of the peroxide remains according to the Peroxide stability test.

14. The oral care composition of claim 1, wherein the composition is a product selected from the group consisting of orally dissolvable films, whitening strips, whitening gels, mouthwashes, toothpastes, or dentifrices.

15. The oral care composition of claim 1, wherein the composition has a viscosity of about 1,000 to about 250,000 cP.

16. The oral care composition of claim 1, wherein the peroxide comprises hydrogen peroxide, urea peroxide, calcium peroxide, sodium peroxide, zinc peroxide, or combinations thereof.

17. The composition of claim 1, wherein the peroxide is hydrogen peroxide.

18. The composition of claim 1, wherein the oral care composition comprises at least one ingredient selected from the group consisting of desensitizing agents, chelating agents, whitening agents, tartar control agents, detergents, adhesion agents, foam modulators, pH modifying agents, mouth-feel agents, sweeteners, flavoring agents, colorants, preservatives, humectant and combinations thereof.

19. The composition of claim 18, wherein the oral care composition comprises the humectant.

20. The composition of claim 19, wherein the humectant comprises glycerin.

21. The composition of claim 1, wherein the peroxide is a liquid formulation.

22. The composition of claim 21, wherein the liquid formulation has a concentration of the peroxide of about 20 wt %, about 25 wt %, about 30 wt %, about 35 wt %, about 40 wt %, about 45 wt % or about 50 wt %.

23. A method of whitening comprising applying to a surface of a mammalian tooth the oral care composition according to claim 1.
